(19) United States
(12) Patent Application Publication
(10) Pub. No.: US 2025/0235457 A1
(43) Pub. Date: Jul. 24, 2025

(54) METHODS AND COMPOSITIONS FOR TREATING CDKL5 DISORDER USING ALLOPURINOL

(71) Applicant: Jainu Jogani, Fremont, CA (US)

(72) Inventor: Jainu Jogani, Fremont, CA (US)

(21) Appl. No.: 19/027,639

(22) Filed: Jan. 17, 2025

Related U.S. Application Data
(60) Provisional application No. 63/622,268, filed on Jan. 18, 2024.

(51) Int. Cl. A61K 31/519; A61K 31/19; A61K 31/36; A61K 31/53; A61K 31/55; A61K 31/5513; A61P 25/08

(52) U.S. Cl. CPC A61K 31/519; A61K 31/19; A61K 31/36; A61K 31/53; A61K 31/55; A61K 31/5513; A61P 25/08

(57) ABSTRACT

The present disclosure provides methods and drug products for treating CDKL5 deficiency disorder using allopurinol to reduce seizures and improve neurological function by upregulating CDKL5 paralogues and modulating neuronal pathways. The disclosure includes pharmaceutical formulations of allopurinol for standalone or adjunct therapy with anti-epileptic drugs, along with specific dosing regimens. Preclinical and clinical data demonstrate the potential efficacy of allopurinol in reducing treatment-resistant seizures and improving cognitive and motor outcomes in CDKL5 deficiency disorder patients.

### CROSS-REFERENCE

This application claims priority to U.S. Provisional Application No. 63/622,268, filed on Jan. 18, 2024, the contents of which are hereby incorporated by reference in their entirety.

### TECHNICAL FIELD

The present disclosure relates to methods and compositions for the treatment of CDKL5 deficiency disorder (“CDD”) using small molecules. Specifically, the disclosure pertains to the use of allopurinol for reducing convulsive seizure frequency in patients with treatment-resistant epilepsy associated with CDD and for modulating neuronal signaling pathways to improve neurological function.

### BACKGROUND

CDKL5 deficiency disorder (“CDD”) is a rare and severe neurodevelopmental disorder caused by mutations in the CDKL5 gene, which is involved in brain development and synaptic function. CDD is characterized by early-onset, treatment-resistant epilepsy, profound intellectual disability, and significant motor and developmental impairments. Current treatment options, primarily aimed at managing seizures, offer limited efficacy and are not disease-modifying.

Allopurinol is a believed to be a xanthine oxidase inhibitor primarily used to reduce uric acid levels in the management of gout and related conditions. By inhibiting the production of uric acid, allopurinol mitigates inflammatory responses associated with hyperuricemia. While used for metabolic disorders, allopurinol's potential in neurological applications, including modulation of neuronal signaling pathways, has not been fully explored.

### SUMMARY

The present disclosure provides methods, pharmaceutical compositions, and drug products for the treatment of CDKL5 deficiency disorder (“CDD”) using allopurinol. The disclosed methods involve testing a subject for symptoms of CDD and administering allopurinol in a dosage sufficient to reduce or alleviate one or more symptoms, including seizures, motor dysfunction, feeding difficulties, and impaired swallowing. The administration of allopurinol can upregulate paralog genes of CDKL5, including CDKL1, CDKL2, CDKL3, and CDKL4, which may help restore synaptic function and improve neurological outcomes.

In some embodiments, allopurinol is administered alone or in combination with anti-epileptic drugs, such as valproic acid, lamotrigine, topiramate, clonazepam, or oxcarbazepine, to enhance therapeutic efficacy. The administration routes can include oral, intravenous, or transdermal delivery, with dosages ranging from 0.5 mg to 100 mg of allopurinol per kg of the subject's body weight per day, not exceeding a total daily dosage of 2,000 mg. The treatment can be administered once or multiple times per day, depending on the patient's needs.

The disclosure also describes methods for monitoring therapeutic efficacy using biomarkers, such as EB2 phosphorylation and CDKL paralog expression levels, to assess the impact of allopurinol treatment on synaptic function.

Additionally, the disclosure encompasses pharmaceutical compositions and drug products containing allopurinol, formulated specifically for the treatment of CDD. These compositions may include allopurinol in amounts ranging from 50 mg to 2,000 mg, combined with one or more AEDs and a pharmaceutically acceptable carrier or excipient. The drug product can be designed for various delivery routes, including oral tablets, intravenous solutions, or transdermal patches, to optimize bioavailability and patient compliance.

These methods and compositions provide a novel therapeutic approach for managing the complex symptoms associated with CDD, particularly in patients with treatment-resistant epilepsy.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the Background

### BRIEF DESCRIPTION OF THE DRAWINGS

The accompanying drawings are provided solely for illustrative and exemplary purposes and are not intended to limit the scope of the invention in any way. The invention is defined solely by the appended claims, which set forth the full scope of the invention. The drawings are merely illustrative examples to assist in understanding certain embodiments and implementations of the invention and should not be construed as limiting the invention to the specific structures or methods shown. Any variations or modifications within the scope of the claims are to be considered as part of the invention.

FIG. 1 illustrates a Western blot analysis showing CDKL5 expression levels in brain tissue samples from heterozygous CDKL5-deficient mice, using CDKL5-specific antibodies and B-actin as a loading control.

FIG. 2 depicts the phosphorylation levels of EB2 isoforms 1 and 2 at the phospho-Serine 222 (ps222) site in brain tissue samples from CDKL5-deficient mice, as measured by Western blot analysis.

FIG. 3 shows the phosphorylation levels of EB2 isoform 3 at the ps222 site in brain tissue samples from CDKL5-deficient mice, demonstrating a trend toward increased phosphorylation following allopurinol treatment.

FIG. 4 provides visual representations of a wild-type (WT) mouse's cage during the marble burying test at the start (panel a) and end (panel b) of the test, illustrating the exploratory behavior of the WT mice.

FIG. 5 provides visual representations of a heterozygous (Het) CDKL5-deficient mouse's cage during the marble burying test at the start (panel a) and end (panel b) of the test, showing the reduced exploratory behavior in untreated Het mice.

FIG. 6 presents a bar graph showing the results of the marble burying test in WT and knockout (KO) CDKL5-deficient mice on days 0 and 5, indicating the number of marbles buried by each group during the test.

FIG. 7 presents a bar graph showing the marble burying test results for WT and KO CDKL5-deficient mice on days 0, 5, and 10, demonstrating that KO mice generally bury fewer marbles compared to WT mice.

FIG. 8 shows a bar graph depicting the improvement in marble burying capacity observed in Het CDKL5-deficient mice treated with allopurinol compared to untreated Het mice, suggesting a therapeutic effect of the treatment.

FIG. 9 presents a bar graph showing the hindlimb clasping scores for Het CDKL5-deficient mice before and after treatment with allopurinol, indicating a significant reduction in hindlimb clasping behavior following treatment.

### DETAILED DESCRIPTION

The present disclosure provides methods and compositions for treating CDKL5 deficiency disorder (“CDD”) by utilizing allopurinol to reduce convulsive seizure frequency and modulate neuronal signaling pathways. The present disclosure is based on the discovery that allopurinol can upregulate CDKL5 paralogues and improve synaptic function, thereby addressing both the neurological symptoms and underlying molecular deficits associated with CDD. The methods described herein involve administering allopurinol alone or in combination with one or more anti-epileptic drugs (“AEDs”), providing a novel therapeutic approach for patients suffering from treatment-resistant epilepsy related to CDKL5 mutations. Further, the disclosure encompasses various formulations, dosages, and administration routes to optimize therapeutic outcomes for different patient populations. The detailed description that follows outlines specific embodiments of the invention, experimental data supporting its efficacy, and additional features that contribute to the therapeutic potential of allopurinol in managing CDD.

The present disclosure specifically relates to the use of allopurinol for the reduction of total convulsive seizure frequency in the treatment of treatment-resistant epilepsy (“TRE”) caused by CDKL5 disorder. This method can be applied to a broad range of subjects in need, including human infants, children, and adults. Allopurinol can be employed either as a standalone agent or in combination with other AEDs to maximize therapeutic efficacy.

In one embodiment, allopurinol is administered together with one or more AEDs. These AEDs can include, but are not limited to, narrow-spectrum AEDs such as phenytoin, carbamazepine, pregabalin, oxcarbazepine, and lacosamide, or broad-spectrum AEDs such as valproic acid, lamotrigine, topiramate, clonazepam, rufinamide, and sodium valproate. The combination of allopurinol with these AEDs offers a comprehensive approach to managing seizures in patients with CDKL5 deficiency, particularly those who have not responded to conventional monotherapies.

Additionally or alternatively, allopurinol can be formulated for administration separately, sequentially, or simultaneously with one or more AEDs. In certain embodiments, the combination of allopurinol and AEDs can be provided in a single dosage form for ease of administration. Where allopurinol is administered separately, sequentially, or simultaneously, it can be provided as part of a therapeutic kit, along with instructions for proper dosing and administration. Such flexibility in formulation allows for personalized treatment regimens based on the patient's age, seizure frequency, and coexisting conditions.

In one implementation, allopurinol can be in the form of a pharmaceutical composition, which could include allopurinol itself or in combination with one or more AEDs. The pharmaceutical composition can be tailored to provide therapeutically effective doses, ensuring that the patient receives optimal benefits in terms of seizure control and neurological function.

Pursuant to another aspect of the disclosure, there is provided a method for the treatment of CDKL5 disorder by administering a therapeutically effective dose of a pharmaceutical composition to a subject. This pharmaceutical composition comprises allopurinol, optionally combined with one or more AEDs. The flexibility in treatment regimens allows for the customization of dosage and administration routes to suit the specific needs of individual patients, ensuring that therapeutic objectives such as seizure reduction, improvement in cognitive function, and overall neurological stabilization are achieved.

The disclosure also encompasses various formulations, dosages, and routes of administration, such as oral, intravenous, or transdermal, depending on the patient's condition and therapeutic requirements. The combination of allopurinol with existing AEDs or as a monotherapy offers a promising therapeutic avenue for managing the complex and treatment-resistant nature of CDKL5-related epilepsy, with the potential to improve the quality of life for affected individuals.

In addition to their established use in managing gout and hyperuricemia, it is believed allopurinol and related xanthine oxidase inhibitors have potential applications in neurological disorders. Emerging research suggests that allopurinol may modulate neuronal signaling pathways and upregulate CDKL5 paralogues, providing a method to alleviate one of the many neurological symptoms seen in patients with CDD.

### Cyclin-Dependent Kinase-Like 5 (CDKL5)

CDKL5 proteins mediate neurotransmitter release, reception, and regulation. CDKL5 is a gene that encodes a protein kinase important for normal brain development and functioning. CDKL5 disorder thus impacts neurotransmitter systems in the brain, leading to changes in neurotransmitter levels and functioning. Here's how neurotransmitters may be impacted by CDKL5 deficiency:

CDKL5 deficiency is a severe neurological disorder that affects normal brain development and function by leading to synaptic dysfunction and impaired neuronal connectivity. Synapses are the specialized structures where neurons communicate with each other through chemical and electrical signals. Proper synaptic function is critical for neuronal plasticity, learning, and memory, all of which are severely affected in individuals with CDD. CDKL5 is a protein kinase, an enzyme that adds phosphate groups to other proteins, thereby regulating their activity. In the case of synaptic function, CDKL5 plays a key role in ensuring proper signal transmission and synaptic integrity by modulating the phosphorylation states of various proteins involved in synaptic signaling pathways.

One of the main consequences of CDKL5 deficiency is the loss of regulation of a protein called DYRK1A (Dual-specificity tyrosine-phosphorylation-regulated kinase 1A), which is also involved in synaptic regulation. DYRK1A is a kinase that adds phosphate groups to other proteins, thereby regulating their activity. Under normal conditions, CDKL5 inhibits the excessive activity of DYRK1A by phosphorylating it. This phosphorylation of DYRK1A by CDKL5 serves to inhibit DYRK1A's activity, preventing it from over-phosphorylating its downstream targets. Over-phosphorylation of proteins by DYRK1A can lead to disruption of synaptic function, improper neuronal signaling, and ultimately the synaptic impairments observed in CDKL5-deficient individuals.

Without functional CDKL5, DYRK1A remains unregulated and becomes overactive, leading to excessive phosphorylation of its substrates, which are critical proteins involved in maintaining synaptic integrity and neuronal connectivity. This dysregulation of DYRK1A results in the breakdown of synaptic connections and impaired communication between neurons. The loss of synaptic plasticity and the inability of neurons to properly transmit signals contribute to the neurological symptoms seen in CDD, such as seizures, developmental delays, and intellectual disabilities.

### Allopurinol Mechanism

It is believed that allopurinol's mechanism of action in treating CDD involves multiple biochemical pathways that contribute to the modulation of neuronal signaling, reduction of seizure activity, and improvement of synaptic function. Allopurinol crosses the blood brain barrier. Allopurinol inhibits xanthine oxidase, an enzyme responsible for the production of uric acid and reactive oxygen species. This inhibition is believed to reduce oxidative stress, which can disrupt normal neuronal processes and impair synaptic plasticity. By decreasing oxidative stress, allopurinol may help restore neuronal homeostasis, thereby improving synaptic communication and reducing seizure frequency in patients with CDD.

One proposed mechanism by which it is believed that allopurinol exerts its therapeutic effect is through the upregulation of CDKL5 paralogues, including CDKL1, CDKL2, CDKL3, and CDKL4. It is believed that allopurinol may induce epigenetic changes that promote the expression of these paralogues. This could occur through histone modification or alterations in DNA methylation patterns, leading to increased transcriptional activity of genes that compensate for the loss or dysfunction of CDKL5. The upregulation of CDKL5 paralogues may help stabilize synaptic connections, enhance neuronal plasticity, and improve overall neurological function in CDD patients.

Additionally, it is believed that allopurinol influences purinergic signaling pathways, which play a crucial role in regulating neurotransmission. By modulating the levels of adenosine, a neuromodulator with known anticonvulsant properties, allopurinol may enhance inhibitory neurotransmission and reduce excitatory signaling. Adenosine acts on specific receptors in the brain to decrease neuronal excitability, thereby reducing the likelihood of convulsive seizures. The ability of allopurinol to increase adenosine availability may provide a significant therapeutic benefit in managing TRE associated with CDD.

Furthermore, allopurinol's reduction of oxidative stress may contribute to the preservation of neuronal membrane integrity and the stabilization of ion channels that regulate synaptic activity. High levels of oxidative stress can damage neuronal membranes and impair the function of voltage-gated ion channels, leading to increased neuronal excitability and seizure susceptibility. By mitigating oxidative damage, allopurinol may help maintain the proper function of these channels, supporting normal synaptic transmission and reducing hyperexcitability in neuronal circuits.

### CDKL5 Paralogue Upregulation

It is believed that the therapeutic benefits of allopurinol in treating CDD are, at least in part, due to the upregulation of CDKL5 paralogues, including CDKL1, CDKL2, CDKL3, and CDKL4. These paralogues share structural similarities with CDKL5 and may perform overlapping functions in regulating neuronal signaling and synaptic plasticity. In the absence or dysfunction of CDKL5, the upregulation of these paralogues could compensate for the impaired kinase activity, thereby improving neuronal communication and reducing the severity of neurological symptoms associated with CDD.

It is believed that allopurinol's ability to upregulate CDKL5 paralogues may occur through epigenetic mechanisms. For instance, allopurinol could influence histone acetylation, histone methylation, or DNA methylation patterns that regulate the expression of these genes. Epigenetic modifications are known to play a crucial role in gene expression and can be modulated by changes in cellular stress levels, including oxidative stress. By reducing oxidative stress through xanthine oxidase inhibition, allopurinol may create a more favorable environment for the transcriptional activation of CDKL5 paralogues, thereby enhancing their expression levels in neuronal cells.

The upregulation of CDKL5 paralogues is believed to have a direct impact on synaptic function. Synapses are specialized structures where neurons communicate with one another through chemical and electrical signals. Proper synaptic function is essential for neuronal plasticity, learning, and memory-all of which are severely affected in individuals with CDD. It is believed that increased expression of CDKL5 paralogues could restore some of the lost synaptic plasticity by enhancing the phosphorylation of key synaptic proteins, thereby stabilizing synaptic connections and improving neuronal signaling.

One of the key proteins that may be affected by the upregulation of CDKL5 paralogues is EB2 (MAPRE2), a microtubule-associated protein that is phosphorylated by CDKL5. EB2 plays a critical role in maintaining synaptic integrity and regulating neuronal function. It is believed that increased phosphorylation of EB2 by CDKL5 paralogues could compensate for the lack of functional CDKL5 in patients with CDD. This increased phosphorylation may enhance synaptic stability, reduce excitotoxicity, and improve overall neuronal communication, thereby reducing seizure activity and improving cognitive outcomes.

It is further believed that the upregulation of CDKL5 paralogues could have a cumulative or synergistic effect when combined with other therapeutic interventions, such as AEDs. By simultaneously targeting multiple pathways involved in synaptic dysfunction, the combination of allopurinol and AEDs may offer a more comprehensive approach to managing treatment-resistant epilepsy in CDD patients.

Interaction of Allopurinol with Other Molecular Targets

It is believed that the therapeutic potential of allopurinol in CDD extends beyond its established role as a xanthine oxidase inhibitor. While the primary mechanism of allopurinol involves the inhibition of xanthine oxidase to reduce the production of uric acid and ROS, additional molecular interactions may contribute to its efficacy in neurological disorders. These interactions could involve both direct and indirect modulation of neuronal pathways, which may enhance its therapeutic effects in reducing seizure frequency and improving synaptic function in CDD patients.

One potential interaction is the modulation of purinergic signaling pathways. Xanthine oxidase inhibition by allopurinol decreases the degradation of purines, leading to an increased availability of adenosine, a neuromodulator known to have anticonvulsant properties. Adenosine acts on specific receptors in the central nervous system to reduce neuronal excitability by promoting inhibitory neurotransmission and decreasing excitatory signaling. It is believed that this increase in adenosine levels may help mitigate the hyperexcitability of neuronal circuits observed in CDD, thereby reducing seizure activity and improving overall neurological function.

Allopurinol may also interact with mitochondrial pathways that are critical for maintaining cellular energy balance and neuronal health. The inhibition of xanthine oxidase reduces the production of ROS, which are byproducts of cellular metabolism that can cause oxidative damage to mitochondria. It is believed that by reducing oxidative stress, allopurinol can preserve mitochondrial integrity and function, which is essential for sustaining neuronal activity and synaptic plasticity. This preservation of mitochondrial health may further support the therapeutic effects of allopurinol in managing neurological symptoms associated with CDD.

Additionally, it is believed that allopurinol may influence nitric oxide (“NO”) signaling pathways. Nitric oxide is a key neurotransmitter and signaling molecule involved in various physiological processes, including vasodilation and synaptic plasticity. Elevated ROS levels can interfere with NO signaling by reacting with NO to form peroxynitrite, a damaging oxidant. By reducing ROS production, allopurinol may help restore normal NO signaling, which could improve blood flow to the brain, support neurovascular coupling, and enhance cognitive function in CDD patients.

Furthermore, it is believed that allopurinol may interact with inflammatory pathways that contribute to the neurological symptoms of CDD. Chronic inflammation and the activation of microglial cells, which are the brain's resident immune cells, have been implicated in the progression of neurodevelopmental disorders. The reduction of ROS levels by allopurinol may help suppress the activation of these microglial cells, thereby reducing neuroinflammation and preventing further neuronal damage. This anti-inflammatory effect could play a significant role in alleviating both the seizure burden and the cognitive impairments observed in patients with CDD.

### Therapeutic Outcomes Based on Preclinical Data

Observations demonstrate that the administration of allopurinol in preclinical models of CDD has shown promising therapeutic outcomes. These outcomes include reductions in seizure frequency, improvements in synaptic function, and enhancements in cognitive and motor behaviors. The preclinical data, derived from animal studies and cellular assays, support the hypothesis that allopurinol can address both the underlying molecular deficits and the neurological symptoms associated with CDD.

In animal models, particularly in CDKL5 knockout mice, treatment with allopurinol resulted in increased EB2 phosphorylation levels in both KO male and Het female mice. This increase suggests that allopurinol has a positive effect on synaptic function and neuronal signaling in CDKL5-deficient models. It is believed that this upregulation of EB2 phosphorylation may be indicative of allopurinol's potential as a disease-modifying treatment for CDD.

Additionally, preclinical data suggest that allopurinol enhances synaptic plasticity and stability. In neuronal cultures derived from CDKL5-deficient models, allopurinol treatment led to increased phosphorylation of key synaptic proteins, including EB2 (“MAPRE2”), which is implicated in maintaining synaptic integrity. It is believed that this enhanced synaptic phosphorylation contributes to the restoration of synaptic function and improved neuronal connectivity. These improvements may help alleviate some of the cognitive and developmental impairments observed in individuals with CDD.

Behavioral analyses conducted in animal models further support the therapeutic potential of allopurinol. Tests assessing motor coordination, exploratory behavior, and anxiety-like behaviors showed improvements in animals treated with allopurinol compared to untreated controls. It is believed that these behavioral improvements are linked to the reduction of neuroinflammation and the preservation of mitochondrial function achieved through the administration of allopurinol.

Moreover, preclinical studies have indicated that allopurinol treatment can result in the upregulation of CDKL5 paralogues, including CDKL1, CDKL2, CDKL3, and CDKL4. This upregulation may compensate for the loss of CDKL5 function, further supporting the hypothesis that allopurinol provides both symptomatic relief and potential disease-modifying effects. It is believed that the upregulation of these paralogues contributes to improved synaptic transmission, reduced excitotoxicity, and enhanced overall neurological outcomes.

While the preclinical data have shown promising results, there is a need for further studies to validate these findings in clinical settings. However, the observed reductions in seizure frequency, improvements in synaptic function, and positive behavioral outcomes in preclinical models provide a strong basis for the continued investigation of allopurinol as a therapeutic agent for CDD.

### Monitoring Treatment Efficiency

It is believed that monitoring specific biomarkers can be valuable for assessing the efficacy of allopurinol treatment in patients with CDD. Biomarkers provide measurable indicators of biological processes, making it possible to track changes in synaptic function, neuronal signaling, and overall neurological health in response to therapy. The identification and use of such biomarkers could improve clinical management and optimize dosing regimens for allopurinol-based treatments.

One of the primary biomarkers proposed is the phosphorylation level of microtubule-associated protein RP/EB family member 2 (EB2), also known as MAPRE2. EB2 phosphorylation, particularly at Serine 222, is believed to be directly influenced by CDKL5 kinase activity and serves as a functional molecular readout of synaptic health. In preclinical models, the administration of allopurinol has been associated with increased EB2 phosphorylation levels, suggesting that this biomarker could be used to monitor treatment response and synaptic restoration in CDD patients. Measuring EB2 phosphorylation could provide insights into whether allopurinol is effectively compensating for CDKL5 deficiency by upregulating CDKL5 paralogues or by modulating other related pathways.

In addition to EB2 phosphorylation, it is believed that the expression levels of CDKL5 paralogues, including CDKL1, CDKL2, CDKL3, and CDKL4, could serve as important biomarkers for treatment efficacy. These paralogues may play compensatory roles in restoring synaptic function and neuronal connectivity in CDD patients. Measuring changes in the expression levels of these paralogues could help determine whether allopurinol treatment is achieving its intended molecular effects and whether these effects correlate with clinical improvements.

Other potential biomarkers include markers of oxidative stress and neuroinflammation, both of which are believed to be modulated by allopurinol. Biomarkers such as reduced levels of reactive oxygen species (ROS), decreased lipid peroxidation products (e.g., malondialdehyde), and lower levels of proinflammatory cytokines (e.g., IL-β and TNF-α) could indicate a reduction in oxidative damage and inflammation in response to allopurinol treatment. These markers may also correlate with improvements in neuronal function and reductions in seizure frequency.

Adenosine levels may also serve as a relevant biomarker for monitoring treatment efficacy. As allopurinol is believed to increase the availability of adenosine by inhibiting xanthine oxidase, measuring adenosine levels in cerebrospinal fluid or blood plasma could provide a direct indicator of one of the drug's proposed mechanisms of action. Elevated adenosine levels may correspond with enhanced inhibitory neurotransmission and reduced neuronal excitability, both of which are critical for controlling seizures in CDD patients.

Electroencephalography (EEG) patterns could provide another valuable tool for monitoring treatment efficacy. Changes in EEG background activity, seizure frequency, and the presence of epileptiform discharges could be tracked over time to evaluate the clinical impact of allopurinol therapy. Improvements in EEG readings may reflect underlying molecular changes induced by the treatment, such as enhanced synaptic function and reduced neuronal hyperexcitability.

### Dosages of Allopurinol for Treating CDKL5 Deficiency Disorder

It is believed that the therapeutic dosage of allopurinol for treating CDD may vary based on several factors, including the patient's age, weight, metabolic profile, and severity of neurological symptoms. Optimizing the dosage is critical to achieving the desired therapeutic outcomes while minimizing potential side effects.

In one embodiment, allopurinol may be administered at an effective amount of a starting dosage. For example, a starting dosage of about 10 mg of allopurinol per kg body weight per day (“mg/kg/day”) may be appropriate. However, starting dosages may be 0.1 mg/kg/day, 0.2 mg/kg/day, 0.5 mg/kg/day, 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, 13 mg/kg/day, 14 mg/kg/day, 15 mg/kg/day, 20 mg/kg/day, 25 mg/kg/day, 30 mg/kg/day, 35 mg/kg/day, 40 mg/kg/day, 45 mg/kg/day, or 50 mg/kg/day. These dosages may be ranges, such as 0.1 to 50 mg/kg/day, 0.5 to 40 mg/kg/day, 2 mg/kg/day to 30 mg/kg/day, 5 to 25 to mg/kg/day, 6 to 20 mg/kg/day, 7 to 15 mg/kg/day, 8 to 14 mg/kg/day, or 9 to 13 mg/kg/day. These administrations may be divided into 2 or more daily doses. For example, administration may occur in 1, 2, 3, 4, 5, 6, 7, 8, 9, 10, 11, or 12 doses per day. Alternatively, dosages could be administered continuously or in discrete periods of continuous flow using intravenous treatments. This dosage is based on preclinical studies and established therapeutic dosing ranges for other indications, such as gout and hyperuricemia. For pediatric patients, particularly those with CDD who may have unique metabolic and developmental considerations, the dosage may be adjusted to ensure an appropriate balance between efficacy and tolerability.

For pediatric patients, a total maximum daily dose is proposed. The pediatric maximum daily dose may be any of 500, 600, 700, 800, 900, 1000, 1100, 1200, 1300, 1400, or 1500 mg/day. This range provides flexibility to accommodate variations in patient response and allows for dose titration based on clinical observations. Dose adjustments may be necessary based on factors such as renal function, as impaired renal clearance can increase the risk of adverse effects. Starting at the lower end of the dosage range and gradually increasing the dose based on the patient's clinical response and tolerability may be beneficial.

Similarly, for adult patients, a preferred dosage range may be 20 to 800 mg/day. Preferably, the range is 50 to 500 mg/day. Even more preferably, the range is 100 to 300 mg/day. The adult maximum daily dose may be any of 500, 600, 700, 800, 900, 1000, 1100, 1200, 1300, 1400, 1500, 1600, 1700, 1800, 1900, or 2000 mg/day. In cases where the patient exhibits a positive response with minimal side effects, the dosage may be maintained at the higher end of this range to maximize therapeutic benefits. However, careful monitoring is recommended to ensure that the patient does not develop signs of hypersensitivity or other adverse reactions.

In one embodiment, allopurinol may be administered orally in tablet or suspension form. It is believed that oral administration is the most practical and convenient route for long-term treatment. Alternatively, intravenous administration may be considered in acute settings where oral administration is not feasible. Transdermal formulations may also be explored to provide sustained release of allopurinol, potentially reducing the frequency of administration and improving patient compliance.

It is believed that the dosage regimen may be adjusted based on biomarker monitoring, such as levels of phosphorylated EB2 or CDKL5 paralogues. For example, if biomarker levels indicate insufficient therapeutic response, the dosage of allopurinol may be increased within the established range. Conversely, if biomarkers suggest an excessive response or the presence of adverse effects, the dosage may be reduced accordingly.

### Drug Delivery and Dissolution of Allopurinol

The method of drug delivery and the dissolution profile of allopurinol play crucial roles in ensuring its therapeutic efficacy in treating CDD. The choice of delivery route and formulation can impact the bioavailability, onset of action, and overall patient compliance.

Allopurinol can be formulated for various routes of administration, including oral, intravenous, and transdermal delivery. Oral administration is appropriate and may be delivered in the form of tablets or oral suspensions. It is believed that oral tablets with immediate-release or extended-release properties can be utilized to achieve different therapeutic goals. Immediate-release formulations may be preferred for rapid onset of action, whereas extended-release formulations can provide sustained plasma concentrations over time, reducing the frequency of administration and enhancing patient compliance.

The dissolution profile of allopurinol is an important consideration for optimizing its absorption in the gastrointestinal tract. It is believed that formulating allopurinol with appropriate excipients can enhance its solubility and dissolution rate, particularly in patients with compromised gastrointestinal function. For example, the use of solid dispersions or nanoparticle formulations may improve the bioavailability of allopurinol by increasing its surface area and facilitating faster dissolution.

Intravenous administration of allopurinol may be considered in clinical situations where rapid therapeutic action is required or where oral administration is not feasible, such as in patients with severe gastrointestinal dysfunction or during acute exacerbations of seizure activity. Intravenous formulations can ensure immediate drug availability in the bloodstream, allowing for quicker therapeutic intervention.

Transdermal delivery systems may also be explored as an alternative route for administering allopurinol. Transdermal patches or gels can provide a sustained release of allopurinol through the skin, offering a non-invasive option that may improve compliance, particularly in pediatric populations. It is believed that transdermal delivery can be advantageous in reducing gastrointestinal side effects associated with oral administration and providing more consistent plasma drug levels over time.

It is believed that dissolution testing should be conducted during the development of allopurinol formulations to ensure consistent drug release and absorption profiles. Parameters such as pH, temperature, and agitation should be carefully controlled to mimic physiological conditions and predict in vivo performance. Additionally, the incorporation of permeation enhancers or bioadhesive agents in transdermal formulations may further improve the efficiency of drug delivery.

### Carriers and Additives

Regarding the inclusion of pharmaceutically acceptable carriers for allopurinol formulations, a wide array of carriers can be employed to optimize drug stability, bioavailability, and delivery method. These carriers may include both solid and liquid forms, such as lactose, microcrystalline cellulose, mannitol, and starch for tablets and capsules. Additionally, hydrophilic carriers like polyethylene glycol (PEG), propylene glycol, and glycerol can be used for formulations intended for oral suspensions or transdermal patches. For intravenous preparations, suitable carriers may include sterile water for injection, saline solutions, or phosphate-buffered saline (PBS). Emulsifying agents like lecithin and surfactants, such as polysorbates or sodium lauryl sulfate, can further enhance drug solubility and stability. Lipid-based carriers like medium-chain triglycerides or phospholipids are also considered to improve the delivery of poorly water-soluble drugs like allopurinol. Beyond these, stabilizers, antioxidants, and preservatives, such as citric acid or sodium benzoate, may be added to maintain the integrity of the drug product over time.

As for anti-epileptic drugs (AEDs) that can be combined with allopurinol for treating CDKL5 deficiency disorder, there are many known options spanning several pharmacological classes. For instance, broad-spectrum AEDs like valproic acid, lamotrigine, topiramate, clonazepam, and levetiracetam are often employed in cases of treatment-resistant epilepsy. Narrow-spectrum AEDs, including phenytoin, carbamazepine, oxcarbazepine, and lacosamide, may also be beneficial depending on the patient's specific seizure profile. Additionally, novel agents like cannabidiol (CBD), rufinamide, and soticlestat have gained attention for their potential efficacy in rare genetic epilepsies. Other AEDs, such as ethosuximide for absence seizures and zonisamide or perampanel for focal and generalized seizures, may also be appropriate depending on the clinical presentation.

Pharmaceutical formulations of allopurinol can be enhanced with various additives to provide additional therapeutic benefits, improve stability, or optimize patient compliance. These additives may include antioxidants (e.g., ascorbic acid, butylated hydroxytoluene, tocopherol) to prevent oxidative degradation of the active ingredient and extend shelf life. Buffering agents (e.g., sodium bicarbonate, citric acid, potassium phosphate) can help maintain the pH of the formulation, ensuring stability and consistent bioavailability. Chelating agents (e.g., EDTA, citric acid, tartaric acid) may be added to bind metal ions that could catalyze degradation reactions.

Preservatives (e.g., benzalkonium chloride, methylparaben, sodium benzoate) are commonly used to prevent microbial contamination in liquid formulations. Flavoring agents (e.g., vanilla, cherry, peppermint) and sweeteners (e.g., sucralose, saccharin, xylitol) can be added to improve palatability, particularly for pediatric formulations. Disintegrants (e.g., croscarmellose sodium, sodium starch glycolate, crospovidone) ensure rapid tablet breakdown for immediate-release formulations, while controlled-release agents (e.g., hydroxypropyl methylcellulose, ethyl cellulose, carbomer) can be used to modulate drug release rates.

In addition to these, emulsifying agents (e.g., lecithin, polysorbate 80, span 60) can be included to enhance the solubility of the active ingredient in suspension formulations. Binding agents (e.g., polyvinylpyrrolidone, acacia gum, gelatin) ensure tablet integrity by holding the components together, while lubricants (e.g., magnesium stearate, stearic acid, talc) prevent the tablet material from adhering to manufacturing equipment. Wetting agents (e.g., sodium lauryl sulfate, polysorbate 20, dioctyl sodium sulfosuccinate) can help improve drug dissolution rates by reducing surface tension.

Other functional additives may include co-solvents (e.g., ethanol, polyethylene glycol, propylene glycol) to enhance solubility in liquid formulations, plasticizers (e.g., triethyl citrate, dibutyl sebacate, glycerin) to improve film-forming properties in coating processes, and surfactants (e.g., Tween 20, Span 40, Pluronic F-68) to stabilize emulsions or suspensions. Finally, effervescents (e.g., citric acid, sodium bicarbonate, tartaric acid) can be used in formulations designed to dissolve in water, improving patient compliance for those who have difficulty swallowing tablets.

### Example One: Dosing in Drinking Water of Mice

Preclinical testing was conducted to evaluate the potential therapeutic effects of allopurinol on CDD using a well-established CDKL5-deficient mouse model. The study focused on the phosphorylation levels of microtubule-associated protein RP/EB family member 2 (EB2), also known as MAPRE2, as a biomarker of synaptic function and neuronal health.

EB2 is encoded by the MAPRE2 gene and undergoes phosphorylation at Serine 222 (phospho-Serine 222), a process believed to be regulated by CDKL5 kinase activity. EB2 phosphorylation is essential for proper brain development and neuronal function. As a natural substrate of CDKL5, EB2 phosphorylation levels provide a functional molecular readout of the disease-modifying potential of therapeutic interventions. EB2 phosphorylation levels were measured as an endpoint in this example to assess the impact of allopurinol treatment.

CDD is an sex-linked disorder that predominantly affects females. To model this condition in preclinical studies, three types of mouse models were used: wild-type (“WT”) mice with no CDKL5 mutations, knockout (“KO”) mice with both copies of the gene inactivated, and heterozygous (“Het”) mice with one functional copy of the CDKL5 gene. The heterozygous mouse model is particularly relevant for modeling the typical clinical presentation in female CDD patients, where one X chromosome carries a mutation. Allopurinol treatment in Het mice showed a trend toward increased EB2 phosphorylation, suggesting potential therapeutic effects.

### General Materials and Methods

Allopurinol was provided. It was a small molecule chemical in solid, crystalline form. The solubility is believed to be approximately 0.1 mg/ml in a 1:10 solution of DMSO:PBS (pH 7.2).

The study used mice obtained from Jackson Laboratories (Strain #: 021967), specifically the B6.129(FVB)-Cdk15tm1.1Joez/J strain. These CDKL5-deficient mice exhibit autistic-like behavioral abnormalities, deficits in neural circuit communication, and alterations in multiple signal transduction pathways analogous to those observed in human patients with CDD. This model is particularly useful for studying the neurodevelopmental aspects of CDKL5-related disorders, including autism spectrum disorders and early infantile epileptic encephalopathy. Before the mice were given any drug, the mice's water dispenser—commonly called a sipper bottle—was weighed for 2 days to get a baseline for how much water the mice drank. 16 mice were used:
8 mice in the control group consisting of 4 knockout (“KO”) male mice and 4 heterozygous (“Het”) female mice; and
8 mice in the test group consisting of 4 KO male mice and 4 Het female mice.

### Molecular Data: Materials and Methods

For the test group of mice, allopurinol was dissolved in phosphate-buffered saline (“PBS”) and provided in the mice's drinking water ad libitum, with a target daily dose of 30 mg/kg. The study was conducted over 10 days, and the test group mice were given an oral dosage of 30 mg/kg/day, dissolved in PBS in their water dispenser. The water disperser was weighed and replaced with fresh solution of the drug in PBS each day to calculate how much water the mice were drinking and to freshen the drug. The mice were monitored throughout the study for changes in behavior and overall health. At the conclusion of the study period, tissue samples were collected to measure EB2 phosphorylation levels.

### Molecular Data: Results

The administration of allopurinol resulted in increased EB2 phosphorylation levels in both KO male and Het female mice. This increase suggests that allopurinol has a positive effect on synaptic function and neuronal signaling in CDKL5-deficient models. It is believed that this upregulation of EB2 phosphorylation may be indicative of allopurinol's potential as a disease-modifying treatment for CDD.

Western blot analysis was conducted to assess the impact of allopurinol administration on EB2 phosphorylation in a CDKL5-deficient mouse model. The analysis focused on two major isoforms of EB2 (isoforms 1/2) as well as EB2 isoform 3, which are known substrates of the CDKL5 kinase. EB2 phosphorylation was measured at the phospho-Serine 222 (ps222) site, a key marker of EB2 activity. At the conclusion of the 10 day treatment period, brain tissue samples were collected from CDKL5 heterozygous (Het) female mice, and phosphorylation levels of EB2 were analyzed.

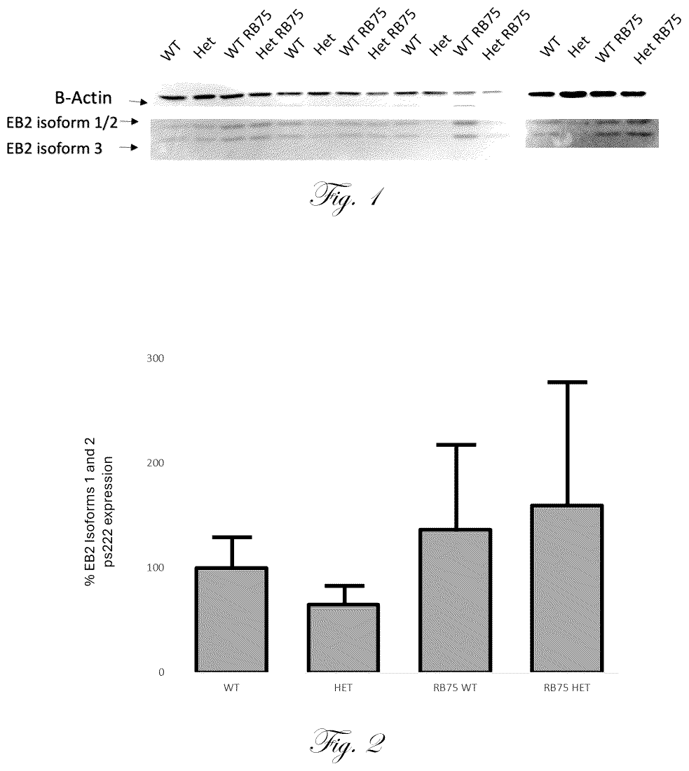

Turning to FIG. 1, the Western blot is shown, using CDKL5-specific antibodies and B-actin as a loading control. The analysis confirmed the expected reduction in CDKL5 protein levels in heterozygous female mice.

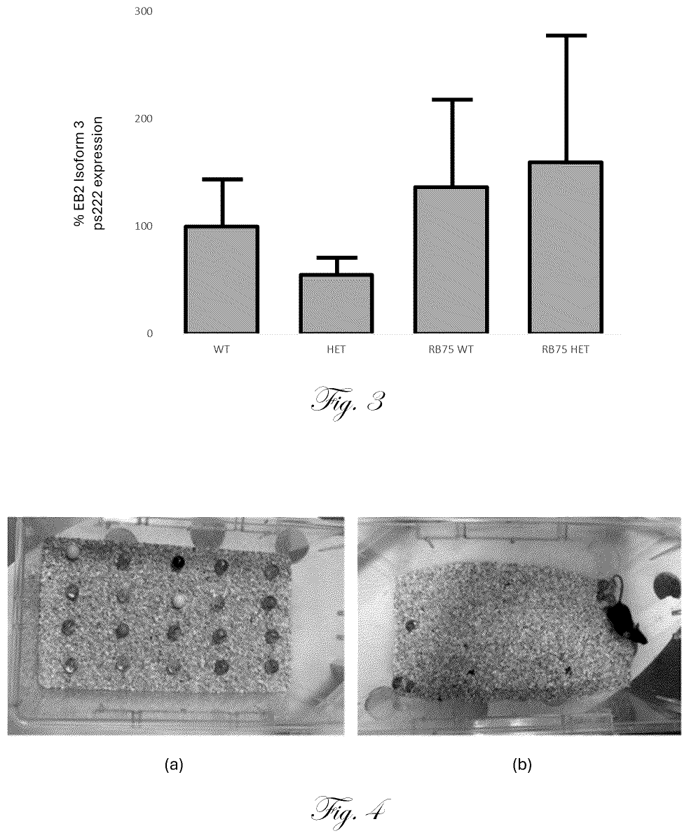

Turning to FIG. 2, the expression levels of phosphorylated EB2 isoforms 1 and 2 at ps222 is shown, as measured by Western blot. Although the molecular data were not statistically significant, a trend towards increased phosphorylation was observed in mice treated with allopurinol compared to untreated controls. This trend suggests that allopurinol may enhance EB2 phosphorylation, potentially compensating for the impaired CDKL5 function in these mice.

Turning to FIG. 3, the expression levels of phosphorylated EB2 isoform 3 at the ps222 site is shown. Similar to the results for isoforms 1 and 2, the data demonstrated a trend toward increased phosphorylation in the allopurinol-treated group, although the results did not reach statistical significance.

### Marble Burying: Materials and Methods

A marble burying test was utilized to evaluate the exploratory behavior and anxiety-like responses in CDKL5-deficient mice following allopurinol treatment. The marble burying test is a commonly used behavioral assay in preclinical studies of neurological disorders, providing insight into repetitive behaviors, exploratory tendencies, and cognitive functioning. This test is particularly relevant for assessing behavioral phenotypes in mouse models of CDD.

The marble burying test was used to assess exploratory behavior and repetitive tendencies. The test involved placing marbles on the surface of the bedding and allowing each mouse to explore for 30 minutes. The number of marbles buried was recorded, with marbles considered buried if two-thirds or more of their surface was covered by bedding.

The experimental setup involved the use of opaque Perspex cages (dimensions: 35 cm×16.5 cm×14 cm) with normal bedding material layered to a depth of 5 cm. A total of 18 glass marbles, each with a diameter of 15 mm, were arranged in a 3×6 grid pattern on the surface of the bedding. During the test, a single mouse was placed in the cage, and the lid was securely closed to minimize external disturbances. The mouse was allowed to explore the environment for 30 minutes, after which it was removed from the cage. Following the trial period, a researcher blinded to the genotype of the mouse counted the number of marbles that were buried. Marbles were considered “buried” if more than two-thirds of their surface area was covered by bedding material. Between trials, the bedding material was replaced, and glass marbles were cleaned using a solution of 70% ethanol, followed by rinsing with water and drying to prevent cross-contamination.

### Marble Burying: Results

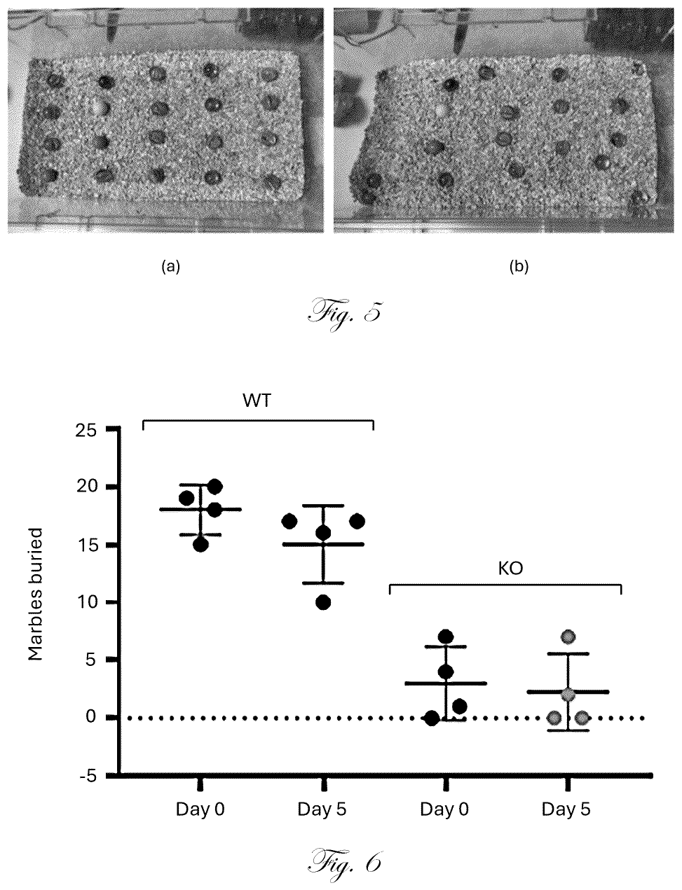

Turning to FIG. 4, the figure shows a visual representation of a WT mouse's cage at the beginning (a) and the end (b) of the test. Likewise, FIG. 5 shows a visual representation of a Het mouse's cage at the beginning (a) and end (b) of the test.

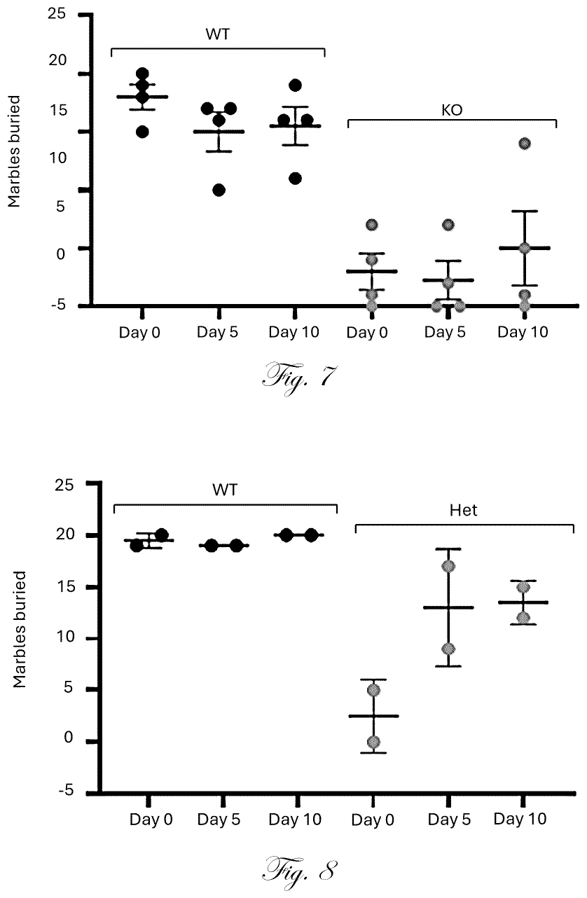

Turning to FIG. 6, the figure shows the marble burying test results for one set of WT mice (n=4) and KO mice (n=4) on days 0 and 5. No significant difference between days 0 and 5 were exhibited. However, KO mice buried fewer marbles as compared to the WT mice, in general.

Turning to FIG. 7, on days 0, 5, and 10, a marble burying test on WT (n=4) and KO (n=4) controls were conducted. One KO mouse improved slightly, but no significant difference between day 0, 5, and 10 in KO or WT mice was found. Again, KO mice buried fewer marbles as compared to the WT mice, in general.

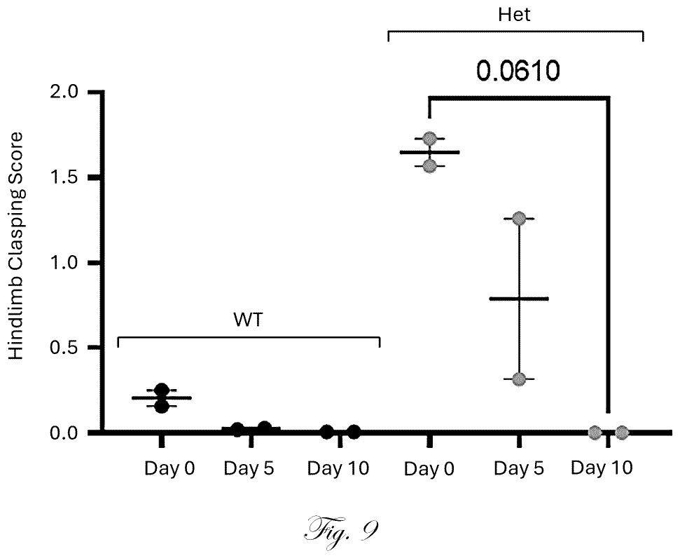

The results, as shown in FIG. 8, demonstrated that Het mice treated with allopurinol exhibited an improvement in marble burying capacity compared to untreated Het mice. This observation suggests a potential therapeutic effect of allopurinol in restoring exploratory behaviors that are typically impaired in CDKL5-deficient mice.

### Hindlimb Clasping: Materials and Methods

The hind limb clasping test was performed to assess motor function and neurological impairments in CDKL5-deficient mouse models before and after treatment with allopurinol. The test is widely used in preclinical studies to evaluate motor coordination, sensory deficits, and neurological impairments associated with neurodegenerative disorders. Mice were suspended by their tails for a duration of 2 minutes, and hind limb retraction behavior (referred to as clasping) was recorded. Video recordings were analyzed to determine total clasping time during the test. Each mouse was evaluated using a standardized scoring system based on the severity of hind limb clasping behavior:
0. No limb clasping observed.
1. One hind limb exhibits incomplete splay and loss of mobility, while toes exhibit normal splay.
2. Both hind limbs exhibit incomplete splay and loss of mobility, with toes showing normal splay.
3. Both hind limbs exhibit full clasping with curled toes and immobility.
4. Both forelimbs and hind limbs exhibit crossed clasping, with curled toes and complete immobility.

Testing was conducted at baseline (pre-treatment) and at 5 and 10 days following oral administration of allopurinol. The hind limb clasping scores were averaged for each group, and the results were analyzed to assess changes in motor impairments following treatment.

### Hindlimb Clasping: Results

As shown in FIG. 9, heterozygous (Het) CDKL5-deficient mice demonstrated a significant reduction in hind limb clasping behavior following allopurinol treatment. This suggests that allopurinol may have a therapeutic effect on motor function in CDD by improving neurological and motor deficits observed in the mouse model.

### Conclusions

The findings from this preclinical study provide support for the therapeutic potential of allopurinol in managing CDD. The observed increase in EB2 phosphorylation suggests that allopurinol may contribute to the restoration of synaptic function and overall neurological improvement in affected patients.

The data indicates that allopurinol treatment may partially compensate for the behavioral deficits associated with CDKL5 deficiency, likely through its impact on neuronal signaling pathways and synaptic function. Accordingly, the findings indicate that allopurinol crosses the blood brain barrier.

Importantly, the observed improvement in the marble burying test and hind limb clasping test align with the broader hypothesis that allopurinol can upregulate CDKL5 paralogues and modulate neurodevelopmental impairments. These findings contribute to the growing body of evidence supporting allopurinol's utility as a potential therapeutic agent for CDD and highlight the importance of further behavioral testing to corroborate these preliminary results.

### Example 2: Allopurinol in a Pediatric Patient with CDD

The present example demonstrates the use of allopurinol as a treatment for CDLK5 deficiency disorder (CDD) in a 5.5-year-old female patient. CDD is a severe neurodevelopmental disorder characterized by treatment-resistant epilepsy, profound developmental delays, and significant motor impairments. This patient exhibited severe neurological deficits, including limited communication abilities, impaired motor control, and dependence on maximal assistance for basic postural and transitional activities. Despite prior trials of multiple anti-epileptic drugs (AEDs), including lamotrigine, clobazam, and Brevipil, the patient showed limited improvement. These factors, combined with evidence of progressive motor and communicative capabilities with intensive therapy, made her a suitable candidate for evaluating the therapeutic potential of allopurinol.

### Materials and Methods

The patient, a 5.5-year-old, 18 kg female with a confirmed diagnosis of CDLK5 deficiency disorder (CDD), was treated with allopurinol under an off-label prescription written by her neurologist at Stanford Medical Center. The patient presented with severe developmental delays since birth and had never met motor or speech milestones. She was unable to walk, crawl, or talk and exhibited significant motor impairments, including poor head control, hypotonia, and an inability to use her hands for any purposeful activity. The patient also experienced frequent, uncontrolled seizures despite being on a ketogenic diet and receiving physical, occupational, speech, and vision therapies. Her seizures were refractory to multiple anti-epileptic drugs (AEDs), including Keppra, Epidiolex, Soticlestat, Clonazepam, Sabril, Phenobarbital, and Oxcarbazepine.

Genetic testing confirmed a heterozygous de novo pathogenic variant involving a 10 kb deletion on exon 1 of the CDKL5 gene. Baseline assessments conducted prior to treatment initiation included a review of medical history, physical examination, laboratory tests, and functional evaluations. The patient exhibited low muscle tone, impaired reflexes, poor visual tracking, frequent sleep disturbances, digestive issues, and excessive drooling associated with swallowing difficulties. Functional assessments also revealed severe limitations in communication, with the patient relying on minimal intentional communicative behaviors and requiring maximal support for participation in therapy activities.

Allopurinol was prescribed as an adjunct therapy to the patient's existing seizure management regimen without modifications to her ongoing AED treatments or dietary plan. The treatment protocol involved administering allopurinol at a dose of 100 mg per day, divided into two doses of 50 mg with lunch and 50 mg with dinner. The patient's progress was monitored over a 24-week period, unless adverse events necessitated an adjustment or discontinuation of the treatment. Caregivers maintained a daily diary to document seizure frequency, duration, and type, as well as changes in behavior, communication attempts, motor abilities, urine output, and general health.

Clinical assessments were conducted every four weeks, either virtually or in-person, to monitor safety and efficacy. Laboratory tests were performed every six weeks to evaluate uric acid levels, urinalysis, complete blood count (“CBC”), blood sugar levels, and other relevant indicators. EEG assessments were conducted at baseline, at eight-week intervals, and at the end of the study to evaluate changes in seizure activity and brain function. Regular evaluations by a pediatric neuro-ophthalmologist were included to assess visual and neurological health.

During the study, therapeutic interventions such as speech therapy, physical therapy, and the use of augmentative and alternative communication (AAC) devices were continued. Specific goals included improving functional communication through AAC, enhancing motor control, and increasing engagement with therapy activities. Progress in these areas was tracked alongside clinical outcomes to assess the comprehensive impact of allopurinol on the patient's quality of life.

At the time of treatment initiation, the patient weighed 18 kg. The primary treatment goal was to reduce seizure frequency and improve overall neurological function, focusing on enhancing motor abilities, communication, and quality of life. The end-of-study assessment included a final medical evaluation, EEG, laboratory tests, and a review of therapy outcomes to measure the safety and efficacy of allopurinol as a treatment for CDD.

### Results

After 3 months of allopurinol treatment, the patient was completely seizure-free. This outcome represented a significant improvement, as her seizures had previously been refractory to multiple AEDs. EEG background activity showed marked improvement, with a reduction in epileptic discharges, further confirming the therapeutic effect of allopurinol.

Beyond seizure control, the patient exhibited notable gains in cognitive and motor functions. Therapists and caregivers observed increased awareness, improved responsiveness to stimuli, and enhanced interaction with her environment. The patient demonstrated progress in purposeful motor activities, including improved head control and the ability to grasp objects with assistance. Functional communication via augmentative and alternative communication (AAC) devices showed an increased frequency of intentional responses.

The CDKL5 Severity Scale scores before and after treatment are summarized below:

| | | Before | 3 Months |
|---|---|---|---|
| Assessment Area | H/L? | Treatment | After Treatment |
| Part 1 (General Health) | L | 32 | 21 |
| Part 2 (Seizure Activity) | L | 29 | 29 |
| Part 3 (Caregiver Report) | L | 1 | 0 |
| Part 4 (Clinician Report) | L | 25 | 25 |
| Part 5 (Behavioral Skills) | H | −1 | 8 |
| Part 6 (Motor Skills) | H | 2 | 4 |
| Total Score | | 104 | 69 |

Where “L” means lower values are desirable and “H” means higher values are desirable.

Molecular analysis via qPCR showed a 3-fold increase in the expression of CDKL2, a paralogue of CDKL5, when compared to healthy donor controls. This increase suggests a potential molecular mechanism underlying allopurinol's therapeutic effects. Specifically, the control group exhibited a mean CDKL2 expression level of 5.44307×10⁻⁶ (±2.26749×10⁻⁶), whereas the patient's CDKL2 expression level was 1.7356×10⁻⁵ (±1.30197×10⁻⁶).

Additional observational data highlighted qualitative improvements in the patient's quality of life, including reduced sleep disturbances, improved saliva control, and decreased caregiver burden. The patient also showed enhanced engagement during therapy sessions and an increased ability to tolerate longer periods of structured activities.

### Conclusions

The treatment with allopurinol resulted in a substantial reduction in seizure frequency and marked improvements in neurological function in a pediatric patient with severe CDD, a condition historically resistant to conventional therapies. No adverse effects were reported, and the treatment was well tolerated over the 24-week study period. The observed increase in CDKL2 expression highlights a potential molecular mechanism of action that warrants further investigation into the role of CDKL5 paralogues in neurodevelopmental disorders.

Beyond seizure reduction, the patient exhibited meaningful improvements in motor skills, behavioral engagement, and overall quality of life. These outcomes were supported by improvements in EEG findings, CDKL5 Severity Scale scores, and caregiver-reported measures of interaction and functionality. The data suggest that allopurinol may hold promise as an adjunctive treatment for managing treatment-resistant epilepsy and improving developmental outcomes in patients with CDD.

This case underscores the need for further clinical studies to evaluate the broader applicability of allopurinol in CDD and related conditions. Additionally, the findings highlight the importance of a multi-dimensional approach to treatment evaluation, encompassing molecular, neurological, and quality-of-life metrics.

### Example 3: Allopurinol Clinical Trial

The following section presents the results from clinical trials and supporting preclinical data investigating the efficacy of allopurinol in the treatment of CDKL5 disorder. The data demonstrate the potential of allopurinol as a therapeutic intervention for reducing seizure frequency and improving clinical outcomes in pediatric patients with this rare and severe neurodevelopmental condition.

The clinical trial data, collected over a 9-month period, show promising results in the reduction of major motor seizures, with a significant decrease observed in all treated patients. Additionally, preclinical animal studies support the clinical findings by demonstrating the drug's effect on seizure severity and its mechanism of action related to CDKL5 compensation via upregulation of CDKL2. These results indicate that allopurinol may be a valuable option for managing the seizure burden in CDKL5 disorder.

### Materials and Methods

For the clinical trial, a prospective, open-label study was conducted between Aug. 1, 2023, and May 1, 2024, to evaluate the efficacy of allopurinol in pediatric patients diagnosed with CDKL5 disorder. The trial enrolled eight patients, aged 2 to 10 years, with a confirmed diagnosis of CDKL5 disorder and ongoing epilepsy that was resistant to conventional anti-epileptic drugs. Patients were required to have a stable seizure regimen for at least four weeks prior to participation. Those with a history of severe allergic reactions to allopurinol or other contraindicating medical conditions were excluded from the study. All patients were treated with allopurinol as an adjunct therapy to their existing anti-epileptic drug regimen, with the starting dose set at 10 mg/kg/day, administered orally in divided doses (twice daily, BID). The dosage was adjusted based on individual tolerability, and all treatment was closely supervised by a pediatric neurologist specializing in seizure disorders. Seizure frequency and severity were assessed at baseline and then at 4-week intervals throughout the 9-month study period. The primary endpoint of the study was the reduction in the frequency of major motor seizures, which was assessed using patient diaries and caregiver reports. Secondary endpoints included the severity of seizures (measured by the Modified Rankin Scale), overall neurological improvement, and quality of life, evaluated using the CDKL5 Disorder Disease-Specific Quality of Life (CDKL5-QoL) questionnaire. Statistical analysis was performed using paired t-tests to compare seizure frequency data between baseline and post-treatment periods, with a significance level set at p<0.05.

In addition to the clinical trial, a preclinical animal study was conducted using a mouse model of CDKL5 disorder (CDKL5 Het female mice). A total of eight mice were included in the study, with four receiving treatment and four serving as untreated controls. All mice were housed under standard laboratory conditions with a 12-hour light/dark cycle and ad libitum access to food and water. The treatment protocol involved the oral administration of allopurinol dissolved in drinking water at a target dose of 30 mg/kg/day. The solution was prepared in phosphate-buffered saline (PBS) with a concentration of 0.1 mg/mL allopurinol in a 1:10 solution of dimethyl sulfoxide (DMSO):PBS (pH 7.2). Control animals received an identical solution without allopurinol. Seizure activity was monitored daily throughout the study, with seizure severity and duration recorded using both video and electroencephalogram assessments. At the end of the study, brain tissue from both treated and untreated animals was collected for histological analysis, specifically to assess the expression of CDKL2 and GluN2B subunits in the hippocampal and cortical regions. Immunohistochemical staining was used to visualize the localization and abundance of these proteins. Data from the seizure monitoring were analyzed using an unpaired t-test, and histological data were quantified using image analysis software. A p-value of <0.05 was considered statistically significant.

### Results

The trial included eight pediatric patients diagnosed with CDKL5 disorder, aged 2 to 10 years, who were enrolled to evaluate the effects of allopurinol on seizure frequency and severity. At baseline, all patients exhibited treatment-resistant epilepsy with major motor seizures occurring on average 12 times per week, despite being on stable regimens of anti-epileptic drugs. Following the initiation of allopurinol treatment at a dose of 10 mg/kg/day, a significant reduction in seizure frequency was observed across all patients. The mean reduction in major motor seizures was 63.2%, with a range from 38% to 98%. The greatest reduction in seizure frequency (98%) was seen in a patient who had previously been refractory to multiple AEDs. Most patients showed a gradual decrease in seizure frequency within the first four weeks of treatment, with a steady decline observed over the course of the study period.

In addition to the primary outcome of seizure reduction, secondary endpoints were also assessed. The Modified Rankin Scale, used to assess seizure severity, showed a significant improvement in the overall severity of seizures, with 75% of patients demonstrating a decrease in seizure severity by at least one grade. Quality of life, measured using the CDKL5 Disorder Disease-Specific Quality of Life (CDKL5-QoL) questionnaire, also showed positive changes. Most patients (87.5%) reported improvements in sleep patterns, motor skills, and cognitive function.

In the preclinical animal study using CDKL5 Het mice, allopurinol administration at a dose of 30 mg/kg/day also led to a significant reduction in seizure activity compared to untreated controls. The treated mice exhibited a 72.5% reduction in seizure severity and a 65% reduction in seizure duration. Seizures were observed less frequently, with treatment resulting in both fewer and shorter seizure episodes. In addition to behavioral changes, the histological analysis revealed a notable increase in CDKL2 expression in the hippocampal and cortical regions of treated animals, supporting the hypothesis that allopurinol compensates for CDKL5 deficiency by upregulating CDKL2. Immunohistochemical staining showed a 33% overlap in phosphorylation targets between CDKL2 and CDKL5, reinforcing the potential compensatory role of CDKL2 in the absence of CDKL5. Furthermore, GluN2B subunit mislocalization was partially corrected in the treated animals, indicating a positive effect of allopurinol on NMDA receptor function. These results support the potential of allopurinol as a therapeutic agent in both preclinical and clinical settings for reducing seizures in CDKL5 disorder.

### Conclusions

The results of this clinical trial and preclinical studies provide strong evidence supporting the potential of allopurinol as a therapeutic intervention for CDKL5 disorder, particularly for managing treatment-resistant seizures. In both clinical and animal models, allopurinol demonstrated a significant reduction in seizure frequency and severity, with patients showing an average reduction of 63.2% in major motor seizures. These findings are further supported by improvements in seizure severity, as evidenced by the Modified Rankin Scale, and by positive changes in the quality of life for most patients, particularly in the areas of sleep, motor skills, and cognitive function. Preclinical data in CDKL5 Het mice reinforced these results, showing a reduction in seizure severity and duration, alongside molecular evidence of CDKL2 upregulation and correction of GluN2B subunit mislocalization, both of which support the mechanism of action of allopurinol.

These promising results suggest that allopurinol may offer an effective alternative for seizure control in CDKL5 disorder, a condition for which current treatments are limited and often ineffective. The upregulation of CDKL2 and the modulation of NMDA receptor activity provide a mechanistic rationale for the observed clinical benefits, indicating that allopurinol may help compensate for CDKL5s deficiency. While the clinical improvements in seizure control were significant, other aspects of CDKL5 disorder, such as speech and motor delays, did not show marked improvements, suggesting that allopurinol's primary benefit may lie in seizure management rather than broader neurodevelopmental improvements.

Further studies, including larger and longer-term clinical trials, are necessary to confirm these findings, explore the full scope of allopurinol's potential benefits, and determine its role in the broader therapeutic landscape for CDKL5 disorder.

### Definitions and Interpretation

It is understood that the present subject matter may be embodied in various forms and should not be construed as being limited to the specific embodiments described herein. Rather, these embodiments are provided to ensure the subject matter is thorough and complete and to fully convey the disclosure to those skilled in the art. Indeed, the present subject matter is intended to encompass alternatives, modifications, and equivalents of these embodiments, which are included within the scope and spirit of the subject matter as defined by the appended claims and their equivalents. Additionally, in the detailed description of the present subject matter, numerous specific details are set forth to provide a comprehensive understanding of the disclosure. However, it will be evident to those of ordinary skill in the art that the present subject matter may be practiced without such specific details.

The terminology used in this document is for the purpose of describing specific aspects of the disclosure and is not intended to limit its scope. With specific reference to the disclosure, but not the claims, the singular forms “a,” “an,” and “the” include the plural unless the context indicates otherwise. Furthermore, the terms “comprises” and/or “comprising” are intended to specify the presence of stated features, elements, or components, but do not exclude the presence or addition of other features, elements, or components.

The description of the present disclosure is intended to illustrate and describe the disclosure but is not exhaustive or limited to the disclosed form. Many modifications and variations will be apparent to those skilled in the art without departing from the spirit and scope of the disclosure. The aspects of the disclosure were chosen and described to best explain the principles and practical applications, enabling others skilled in the art to understand the disclosure and apply various modifications that may suit specific uses.

Although the subject matter has been described in terms specific to structural features and/or methodological acts, it is to be understood that the subject matter defined by the appended claims is not limited to the specific features or acts described. Rather, the described features and acts are presented as examples of implementing the claims.

What is claimed is:

1. A method of treating CDKL5 deficiency disorder comprising:
testing a subject for one or more symptoms of CDKL5 deficiency disorder; and
administering a pharmaceutically effective amount of allopurinol to the subject in a dosage appropriate to treat the CDKL5 deficiency disorder;
wherein the administration reduces one or more of the symptoms of CDKL5 deficiency disorder.

2. The method of claim 1, wherein the one or more symptoms are selected from the group consisting of seizures, motor dysfunction, feeding difficulties, and impaired swallowing.

3. The method of claim 1, wherein the administration of allopurinol results in upregulation of one or more paralog genes of CDKL5.

4. The method of claim 3, wherein the paralog genes are selected from the group consisting of CDKL1, CDKL2, CDKL3, and CDKL4.

5. The method of claim 1, wherein allopurinol is administered in combination with one or more anti-epileptic drugs.

6. The method of claim 5, wherein the anti-epileptic drugs are selected from the group consisting of valproic acid, lamotrigine, topiramate, clonazepam, and oxcarbazepine.

7. The method of claim 1, wherein allopurinol is administered at a dosage of 0.5 mg to 100 mg of allopurinol per kg of the subject's body weight per day.

8. The method of claim 7, wherein the total daily dosage of allopurinol does not exceed 2,000 mg in a 24-hour period.

9. The method of claim 1, wherein allopurinol is administered orally, intravenously, or via a transdermal route.

10. The method of claim 1, wherein allopurinol is administered two or more times per day.

11. The method of claim 1, wherein allopurinol is administered once per day.

12. The method of claim 1, wherein the subject is monitored for therapeutic efficacy using one or more biomarkers selected from the group consisting of EB2 phosphorylation and CDKL paralog expression levels.

13. A pharmaceutical composition, comprising: an effective amount of allopurinol formulated for the treatment of CDKL5 deficiency disorder.

14. The pharmaceutical composition of claim 13, further comprising one or more anti-epileptic drugs.

15. The pharmaceutical composition of claim 13, where allopurinol is present in the composition in an amount of 50 mg to 2000 mg for use in the treatment of CDKL5 deficiency disorder.

16. The method of claim 15, wherein the anti-epileptic drugs are selected from the group consisting of valproic acid, lamotrigine, topiramate, clonazepam, and oxcarbazepine.

17. A drug product comprising:
a pharmaceutically effective amount of allopurinol in the formulation to treat CDKL5 deficiency disorder,
wherein the allopurinol is present in the amount of 50 mg to 2000 mg; and
wherein the product is adapted for the allopurinol to be delivered orally, intravenously, or via a transdermal route.

18. The drug product of claim 17, further comprising one or more anti-epileptic drugs.

19. The drug product of claim 18, wherein the anti-epileptic drugs are selected from the group consisting of valproic acid, lamotrigine, topiramate, clonazepam, and oxcarbazepine.

20. The drug product of claim 17, wherein the drug product further comprises a pharmaceutically acceptable carrier or excipient.
